(19) United States
(12) Patent Application Publication
(10) Pub. No.: US 2019/0302027 A1
(43) Pub. Date: Oct. 3, 2019

(54) METHOD AND APPARATUS FOR DETERMINING SOLIDS CONTENT IN A LIQUID MEDIUM

(71) Applicant: BRÄNNSTRÖM GRUPPEN AB, Göteborg (DE)

(72) Inventor: Roland BRÄNNSTRÖM, Göteborg (SE)

(73) Assignee: BRÄNNSTRÖM GRUPPEN AB, Göteborg (SE)

(21) Appl. No.: 16/317,205

(22) PCT Filed: Jun. 20, 2017
(86) PCT No.: PCT/SE2017/050675

§ 371 Date: Jan. 11, 2019

(30) Foreign Application Priority Data
Jul. 14, 2016 (SE) 1651057-0

(51) Int. Cl. G01N 21/65; G01N 21/53

(52) U.S. Cl. CPC G01N 21/65; G01N 33/18; G01N 21/53

(57) ABSTRACT

Disclosed is an apparatus for determining solids content of a liquid medium of a test sample. The apparatus comprises one or more light source for directing a light beam of a first wavelength range towards the test sample and one or more detector for collecting irradiation emitted from the liquid medium of the test sample as a result of the light beam directed towards the test sample, the irradiation being collected at one or more second wavelengths that are characteristic for the liquid medium. The detector is further arranged for measuring an intensity of the irradiation collected at the one or more second wavelengths. The apparatus further comprises a determining unit for determining the solids content of the liquid medium based on the measured intensity of the irradiation collected at the one or more second wavelengths. By determining solids content based on irradiation emitted from the test sample at the second wavelength that is characteristic for the liquid medium and different from the first wavelength, a good determination could be achieved for very turbid samples.

### TECHNICAL FIELD

The present disclosure relates generally to a method and an apparatus for determining solids content in a liquid medium. Solids content of a liquid medium can be linked to turbidity, content of suspended solid or concentration of a single solute in a liquid medium of a test sample.

### BACKGROUND

Knowing the solids content of a liquid or slurry is an important step for understanding physical properties of the liquid or slurry. In the following, the term “liquid” comprises both liquids and slurries. Solids are the portion of a liquid that is left when water (or other liquid medium such as an alcohol e.g. methanol) is removed. The amount of solids in waste water and manure affects nutrient content, treatment processes and handling procedures. There are many application areas where it may be of interest to determine solids content, e.g. turbidity, of a liquid medium. One such application area is automatic control of polymer dosage for sludge dewatering in wastewater plants. For achieving correct control of polymer dosage the solids content of the sludge needs to be accurately determined. Also, a solids content sensor for such an application area should have a short response time and be maintenance free.

There are prior art turbidity sensors developed as probes which are arranged to be inserted into the liquid. Such a probe emits light from a light source through a window into the liquid and determines turbidity of the liquid based on elastically scattered light in an angle of e.g. 90 and 135 degrees from the direction of the emitted light. As such a probe is inserted into the liquid, the probe needs to be cleaned in regular intervals so that for example the windows through which the emitted light is exiting and through which reflected light is entering do not become dirty and influence the measurement results. Further, with such probes it is difficult to determine solids content or turbidity for very turbid liquids as the light that travels from the light source through the liquid before being received at the receiver of the probe tends to be very much dampened at high turbidities before it is received at the receiver. Further, there are other prior art turbidity measurement apparatuses that are arranged outside of the liquid, Such turbidity measurement apparatuses sends light towards the liquid and detects elastic reflections of the sent light onto a detector of the apparatus, Such apparatuses do not need to be cleaned as often as the probes. However, there is difficult to determine solids content for very turbid samples with such apparatuses, as is further described in the detailed description. Consequently, there is an interest of a turbidity sensor/apparatus that can achieve a good determination of turbidity at a larger measurement range than the prior art, e.g. at higher turbidities than what is possible with prior art sensors/apparatuses. Also, it would be beneficial with a turbidity sensor/apparatus that is more or less maintenance free.

### SUMMARY

It is an object of the invention to address at least some of the problems and issues outlined in this disclosure. It is an object of embodiments of the invention to reliably determine the solids content of a liquid medium with high precision. It is another object of embodiments of the invention to reliably determine the solids content of the liquid medium at very high turbidity levels. It is possible to achieve one or more of these objects and others by using a method and an apparatus as defined in the attached independent claims.

According to an aspect, an apparatus is provided for determining solids content of a liquid medium of a test sample. The apparatus comprises one or more light source for directing a fight beam of a first wavelength range towards the test sample and one or more detector for collecting irradiation emitted from the liquid medium of the test sample as a result of the light beam directed towards the test sample, the irradiation being collected at one or more second wavelengths that are characteristic for the liquid medium. The one or more detector is further arranged for measuring an intensity of the irradiation collected at the one or more second wavelengths. The apparatus further comprises a determining unit for determining the solids content of the liquid medium based on the measured intensity of the irradiation collected at the one or more second wavelengths.

By measuring the intensity of irradiation collected at a wavelength that is different from the wavelength of the incident light and at the same time at a wavelength that is characteristic for the liquid medium, such as Raman reflection, instead of measuring on elastic reflected light as in prior art, it is possible to determining solids content for liquids having a higher turbidity than what is possible for measurements on elastically reflected light.

According to an embodiment, the apparatus is arranged such that the irradiation collected by the one or more detector is emitted from a first area of the test sample and which first area is at least partly illuminated by the light beam of the light source. When trying to detect emitted Raman light with optical-based turbidity devices at an angle of e.g. 90 degrees compared to the incident light, some of the incident and emitted light will be absorbed in the test sample before it is detected as reflected light, as the emitted and reflected light has to travel a not insignificant distance in the test sample before it is detected. This fact makes detection at very high turbidity levels difficult, as light travels much less distances until it is absorbed in samples with high turbidity than in samples with low turbidity. However, by instead detecting irradiation emitted from areas that are at least partly covered by the illuminated area, as in the present invention, the reflected light can be strong enough to be detected, also for test samples having high turbidity.

The areas from which irradiation is emitted and collected by the one or more detector are surface areas of the test sample. The illuminated area is a surface area. Further, the surface areas may be seen as envelope surfaces of the test sample. For example, the illuminated area may be seen as the illuminated part of the total envelope surface of the test sample. The apparatus may be implemented in different ways to achieve that the areas of the test sample from which irradiation collected by the detector is emitted, is at least partly illuminated by the light beam of the light source. Different possible examples of apparatus implementations are shown in the appended figures. According to an embodiment, the apparatus is arranged with light directing devices and light focusing devices so as to achieve that the irradiation collected by the one or more detector is at least partly illuminated by the light source. For example, irradiation directing devices such as prisms and lenses may be used to see to that it is irradiation reflected from a certain area that is received at the detector. In a similar way, the light emitted by the one or more light source may be focused by light directing devices such as lenses towards an area of the test sample to be illuminated so as to achieve an efficient and strong enough light onto a specified area of the test sample. There may be more than one light source directing light beams towards the samples. There may also be more than one light detector for detecting reflected irradiation from the test sample.

According to an embodiment, the liquid medium is water and the second wavelength characteristic for the liquid medium may be a wavelength characteristic for Raman reflection of water.

According to an embodiment, the light source is arranged to illuminate an illumination area of the test sample, and the one or more detector is arranged such that the first area and the second area are substantially covered by the illumination area. By illuminating an area of the test sample that covers the areas from which irradiation is collected by the detector, emitted edge effects occurring at edges between illuminated and not illuminated areas are lowered. Such edge effects may have negative impact on the accuracy of the measurements of emitted irradiation. According to another embodiment, the illumination area not only covers the first area and the second area but also is larger than these areas. Hereby, edge effects are lowered even more.

According to another embodiment, the apparatus is arranged so that there is an angle between the light beam directed towards the test sample and the irradiation emitted from the test sample that is received by the one or more detector that is lower than 45 degrees, preferably lower than 10 degrees, most preferably approximately zero degrees. Hereby it is achieved that the first area of the test sample, from which irradiation collected by the detector is emitted, is at least partly illuminated by the light beam of the light source. Further, by having an approximately zero degree angle between incident and emitted light, as in the most preferable embodiment, the illuminated volume of the test sample is substantially the same as the volume from which emitted irradiation is detected by the detector, Hereby, even more accurate measurement values can be achieved. An apparatus that is arranged in this way is described in FIG. 4. However, other similar apparatuses may achieve the same function, such as polarizing beam splitters being illuminated with monochromatic laser light. Responding irradiation in form of scattered depolarized light resulting from inelastic scattering will mainly go straight through the beam splitter to the irradiation detector.

According to another embodiment, the one or more detector is further adapted to collect irradiation and measure intensity of the irradiation at the second wavelength, the irradiation being emitted from a first reference sample comprising a known solids content of the liquid medium. Further, the determining unit is adapted to determine the solids content of the liquid medium of the test sample based on the measured intensity of the irradiation collected at the second wavelength from the first reference sample as well as from the test sample. By taking into account measured intensity values for a reference sample having a known solids content in liquid medium, the apparatus can be calibrated, thereby improving the accuracy of the turbidity/solids content determination.

According to another embodiment, the one or more detector is further adapted to collect irradiation and measure intensity of the irradiation at the second wavelength, the irradiation being emitted from a second reference sample comprising a known solids content of the liquid medium different from the known solids content of the first reference sample. Further, the determining unit is adapted to determine the solids content of the liquid medium of the test sample based on the measured intensity of the irradiation collected at the second wavelength from the first reference sample, from the second reference sample as well as from the test sample. The first reference sample or the second reference sample can be a clean sample, i.e. a reference sample having zero solids content. The other one of the first reference sample and the second reference sample may have a solids content in a turbidity region where measured intensity of the second wavelength has a substantially linear relationship to the turbidity value.

According to an embodiment, the apparatus further comprises a temperature sensor for detecting the temperature of the test sample. Further, the determining unit is arranged for determining the solids content of the liquid medium of the test sample further based on the detected temperature of the test sample. There is a temperature dependency between detected irradiation levels and the turbidity of the medium. At room temperature, Raman scattering at a larger wavelength than the wavelength of the incident light, i.e. at a lower energy level, so called Stoke Raman, is much more common than Raman scattering at a shorter wavelength than the wavelength of incident light, so called anti-Stokes Raman. As the temperature increases, the Stokes Raman is decreased and the anti-Stokes Raman is increased. Knowledge of this temperature dependency can be used so that the actual temperature of the test sample is taken into consideration when determining the turbidity in the medium of the test sample.

According to another aspect, a method is provided for determining a solids content in liquid medium of a test sample. The method comprises directing a light beam of a first wavelength range towards the test sample, collecting irradiation emitted from the liquid medium of the test sample as a result of the light beam directed towards the test sample, the irradiation being collected at one or more second wavelengths that are characteristic for the liquid medium, and measuring an intensity of the irradiation collected at the one or more second wavelengths. The method further comprises determining the solids content of the liquid medium of the test sample based on measured intensity of the irradiation collected at the one or more second wavelengths.

Further possible features and benefits of this solution will become apparent from the detailed description below.

### BRIEF DESCRIPTION OF DRAWINGS

The solution will now be described in more detail by means of exemplary embodiments and with reference to the accompanying drawings, in which:

FIG. 1 is a schematic block diagram of an optical detector system in which the present invention may be used.

FIG. 2 is a schematic block diagram illustrating penetration depth in an optical detector system as in FIG. 1 for light falling onto a sample having high turbidity in relation to a sample having low turbidity illustrated in FIG. 1.

FIG. 3a is an x-y diagram showing absorption coefficient proportional engineering unit, NTU, which is a measure of the turbidity, at its x-axis, and measured intensity of inelastic response of liquid medium at its y axis.

FIG. 3b is an x-y diagram showing volume share at its x-axis and measured intensity of inelastic response of liquid medium at its y axis for a number of substances with different absorption coefficients.

FIG. 4 is a schematic block diagram of another optical detector system in which the present invention may be used.

FIG. 5 is an x-y diagram showing turbidity on the x-axis and for the dot line intensity of elastic response on the y-axis and for the solid line inelastic response on the y-axis

FIG. 6a is an x-y diagram showing turbidity on the x-axis in relation to measured intensity on the y-axis for an inventive apparatus in relation to prior art, wherein the y-axis has a linear scale.

FIG. 6b another x-y diagram showing turbidity on the x-axis in relation to measured intensity on the y-axis for an inventive apparatus in relation to prior art, wherein the y-axis has a logarithmic scale.

FIG. 7 is a flow chart illustrating a method according to an embodiment of the invention.

### DETAILED DESCRIPTION

Briefly described, a solution is provided to optically determine the solids content of a liquid medium or the turbidity of the liquid medium of a test sample, which solution is especially adapted for determining the turbidity or solid content in test samples that has a high irradiation absorption coefficient. A high irradiation absorption coefficient signifies a short penetration depth for the irradiation, e.g. light, which signifies that the test sample has a high turbidity. The turbidity or solids content of a liquid medium is determined by an apparatus comprising a light source arranged to direct irradiation in the form of light of a first wavelength range towards the test sample, and a detector for detecting intensity of backscattered irradiation from the test sample at a second wavelength characteristic for the liquid medium, e.g. water, as a result of the light directed towards the test sample. The turbidity or solids content of the liquid medium is then determined based on the detected intensity of backscattered irradiation at the second wavelength. By determining solids content or turbidity of a liquid medium based on backscattered irradiation as a result of inelastic scattering, i.e. as a result of reactions with the liquid medium, instead of measuring based on scattered elastic irradiation, i.e. reflections at the same wavelength as the incident light, more precise values for solids content or turbidity can be achieved, especially for test samples that has high irradiation absorption coefficient, i.e. very turbid test samples.

According to an embodiment, for being able to get enough backscattered irradiation also from test samples that has high irradiation absorption coefficient, i.e. short light penetration depth, the irradiation detected by the detector is emitted from an area of the test sample that is illuminated by the light source. Hereby, the turbidity or the solids content of the liquid medium can be determined also for test samples having a very short irradiation penetration depth.

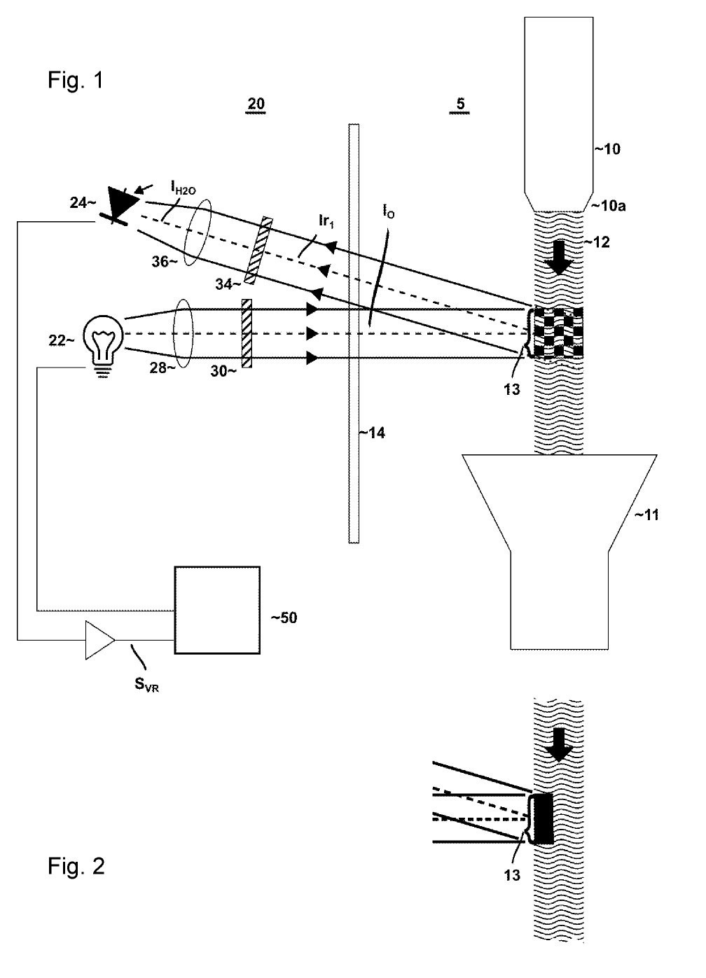

An embodiment of an apparatus for determining solids content of a liquid medium is described in FIG. 1. The liquid medium has a certain turbidity or solids content, due to e.g. particles of any kind that are situated in the liquid medium, originating from e.g. a substance in the liquid medium. The substance may be a liquid substance. In the following example the substance will be exemplified by oil and the liquid medium will be exemplified by water. The substance may be a palette of different oils with the characteristic of absorbing light. A test sample 12 comprising turbid water may be led through a wet part 5 of the apparatus, the wet part comprising a pipe 10 and a funnel 11. The pipe 10 may end in a tap 10a spaced apart from and arranged above the funnel 11 so that the water-oil mixture of the test sample falls in a free-falling jet from the pipe 10 until it is received in the funnel 11 arranged below the pipe. A light source 22 of a detecting part 20 of the apparatus is arranged so that light 10 emitted from the light source will enter the test sample at a light-entering area 13 where the test sample falls in a free-falling jet from the pipe towards the funnel. In a not shown alternative, the sample may be led in a pipe also when it passes the light-entering area. In this alternative, the pipe 10 will have a transparent part through which the light beam may pass and come into contact with the sample. However, by arranging the wet part with a pipe and a funnel spaced apart so that the sample will fall in a free falling jet at the light-entering area, no such transparent part is needed, and the risk that this transparent part becomes dirty after being used some time is avoided. The light emitting and detecting part 20 comprises a collimator having at least one first convex lens 28 that focuses part of the light emitted by the light source 22 towards the light-entering area 13 of the wet part and a bandpass filter 30 that only lets a first wavelength range of the emitted light through, which first wavelength range is to be sent towards the light-entering area.

The light emitting and detecting part 20 comprises, except for the already mentioned light source 22, also a detector 24 for detecting an intensity of irradiation at a second wavelength characteristic for water reflection, e.g. Raman reflection of water. The second wavelength is different from the first wavelength of the light entering the test sample at the light-entering area. The detector may be a photo diode. The light source 22 may be a Light Emitting Diode, LED. The emitted light may be in the ultraviolet, UV, range. The detecting part 20 may further comprise a protection window 14 for letting through light/irradiation and preventing dirt to enter the detecting part 20. The protection window is spaced apart from the test sample.

As the incoming light I₀ falls onto the test sample 12, the liquid medium will absorb a fraction of the incoming light for every slab of test sample. A slab could be seen as an infinitesimally thin part of the test sample that the light penetrates through. A fraction of the absorbed light will scatter back as the result of inelastic scattering, i.e. that the scattered particles have an energy that is lower and/or possibly higher than the energy of the photons falling onto the test sample. The inelastic scattered irradiance is characteristic for the liquid medium. Fluorescence and Raman scattering are results of such inelastic scattering. The inelastic scattered irradiance is omnidirectional. A part of the inelastic scattered irradiance will be reflected back towards detector 24. Before falling onto detector 24, the reflected irradiance Iᵣ₁ passes through a bandpass filter 34 that only lets through wavelengths characteristic for inelastic scattering of water, such as the Raman reflection of water. Hereby, elastic scattering wavelengths as well as other inelastic scattering wavelengths are filtered out. The irradiance of the wavelengths for inelastic scattering of water is further received by an objective 36 comprising one or more lenses to concentrate the irradiance towards the photo diode 24 that determines the intensity of the inelastic scattered irradiance of water, e.g. the Raman reflection. The intensity may be determined by determining an energy level or power level of the received irradiance. The detector is positioned so that the intensity resulting from irradiance due to inelastic scattering of water I_{H20} it receives is emitted from an area of the test sample that is covered by the light-entering area 13. The optics of the apparatus, i.e. the objective 36 of the detector is arranged so that it is the scattered irradiance received from an area of the test sample covered by the light-entering area 13 that is received by the photo diode 24.

When using the apparatus of FIG. 1 on a test sample, and when the intensity of inelastic scattered irradiance of water has been measured by detector 24, information on the measured intensity is sent to a determining unit 50 that determines the turbidity or solids content based on the received information. The measured intensity may be a level of signal strength, power or energy.

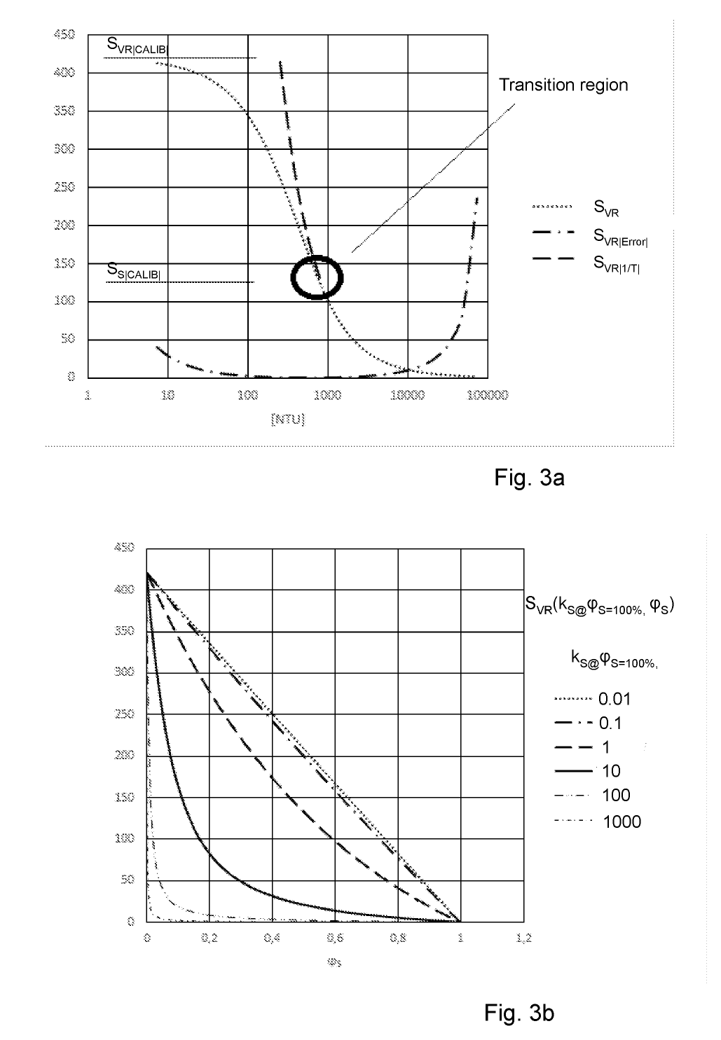

The slabs of the test sample may also contain other light absorbers than the liquid medium itself, such as particles that scatter light, content of suspended solid or liquid substances. These absorbers will reduce the light penetrating each slab resulting in less incoming light to the next slab in the light direction. The inelastic scattered irradiance is omnidirectional. A fraction of the inelastic scattered light in a slab will get the propagation direction back towards the surface where it came from. On the way back it will once more pass all slabs being subject to a corresponding procedure as on the way in. The filter 34 is arranged so that only the inelastic scattered light from the liquid medium will pass the filter 34, so as to filter out other wavelengths that would disturb the measurement. The filtered light will be detected by detector 24. Maximum light on detector 24 is received when there is no other light absorbers than the liquid medium. For water being the liquid medium and for a geometrical path length of the test sample that is significantly shorter than the inverse of the absorption coefficient of the test sample, the maximum light on detector 24 will be limited by the geometrical path length of the test sample. Therefore, the measured intensity of water Raman S_{VR} at detector 24 tends to flatten out towards lower turbidity measures. The measured intensity of water Raman S_{VR} is shown as a dotted curve in the x-y diagram of FIG. 3a, The flattening out towards lower turbidity measures can be seen at the left side of the x-y diagram of FIG. 3a. As the content of other light absorbers is increased, i.e. as the turbidity of the test sample increases, the light on detector 24 will decrease. For a certain amount of absorbers, here called a transition region, and for a higher amounts of absorbers than in the transition region, the inventor has observed that the test sample path length will no longer conform the limitation of scattered light on detector 24. The transition region is marked in the diagram of FIG. 3a, which will be further described further down. Further increase of absorbers resulting in a penetration depth being much shorter than the test sample path length, see FIG. 2, will furthermore reduce the light on detector 24, The absorbers only, will now confirm the limitation of light on detector 24. The ratio of light at the detector 24 will now be inversely proportional to the sample's total absorption coefficient, See the right side of diagram 3a, to the right of the transition region.

Given that all properties, such as intensity of the LED, attenuation of filters, optical properties of lenses, test sample, windows, etc., test sample path length, detector sensitivity, absorption coefficients of liquid medium as well as other absorption coefficients, Raman, fluorescence and elastic scatter efficiency, etc, are known, calibration of the measuring system is not required. However a calibration process simplifies interpretation of the results produced by the measuring apparatus.

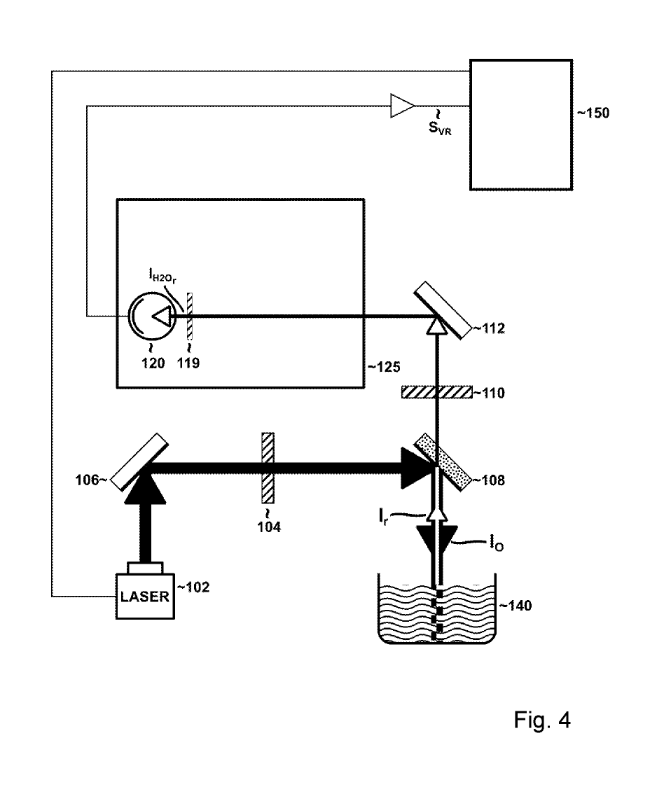

In the following, an embodiment of a two-stage calibration process is described. In stage one, a liquid medium without absorbers is inserted as a first reference sample in the apparatus of FIG. 1 or FIG. 4. The light source is switched on and the detector 24 measures the amount of scattered light it receives and sends the measurement value, called S_{VR|CALIB}, to the determining unit 50 that stores S_{VR|CALIB|} and possibly displays the value on its display. The total impact on the measuring apparatus of the listed properties are now known except for the properties of the absorbers that gives rise to the turbidity the apparatus is dedicated to measure.

In stage two, a second reference sample having a known turbidity that lies within or to the right of the transition region of FIG. 3a is inserted in the apparatus of FIG. 1 or FIG. 4. The light source is switched on and the detector 24 measures the amount of scattered light it receives and sends the measurement value, called S_{S|CALIB|}, to the determining unit 50 that stores S_{S|CALIB|} and possibly displays the value on its display. As the apparatus now knows measured scattered light and the corresponding turbidity for two points of the diagram of FIG. 3a, the total transfer function for the apparatus between light at detector 24 and the turbidity of the test sample can now be estimated by the determining unit 50.

The solids content/turbidity is now determined based on the measured intensity S_{VR} of the second wavelength of the test sample, the measured intensity S_{VR|CALIB|} without absorbers, i.e. for solids content=0, and the measured intensity S_{S|CALIB|} for a known solids content/turbidity=x.

One of the most important properties of embodiments of the present invention is its ability to determine turbidity at liquid mediums with high amount of absorbers. With this in consideration, it is in principle only necessary to calibrate according to stage two, i.e. for measured intensity for a known solids content x. The drawback is that it is difficult to determine the transition region without the knowledge of stage one. Stage one is also provided in order to calculate the remaining impact that the test sample path length has on stage two calibration, Selecting a stage two calibration point well to the right of the transition region will solve this issue. However the user need to keep in mind that calibrating stage two with very high amount of absorbers may reduce the resulting accuracy due to the decreasing amount of light on detector 26.

The test sample path length has an increasing impact on the light detected on detector 24 for decreasing amount of absorbers in the test sample. The transition region in FIG. 3a is selected in such a way that for a certain amount of absorbers in the test sample the mentioned impact is in the same order as the precision of the detector. The transition region detector precision is approximately 1% at the transition region of an apparatus having properties according to FIG. 3a. The optimal transition region for units with better signal to noise properties is towards right with higher ratio between S_{VR|CALIB|} and S_{S|CALIB|}.

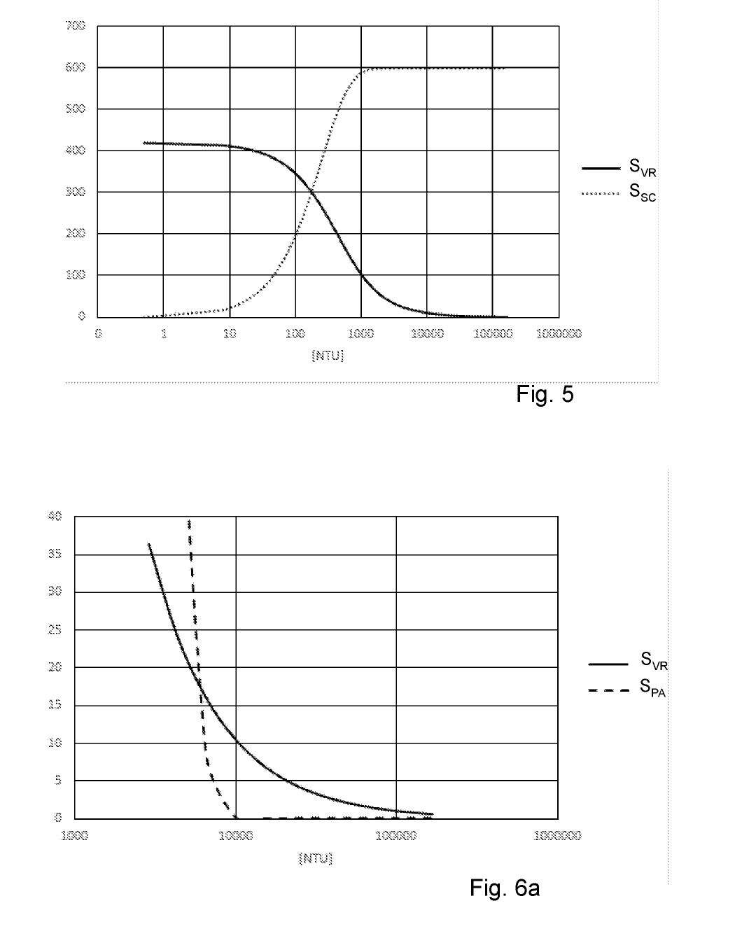

If an apparatus similar to the apparatus of FIG. 1 or FIG. 4 would be used for determining solids content of a liquid medium of a test sample based on detected reflected incoming light, i.e. elastic reflections basically in the same wavelength area as the incoming light, the intensity of the detected reflections would have a curve S_{SC} as shown in the dotted line of FIG. 5, As could be seen in FIG. 5, the detected elastically reflected signal intensity S_{SC} increases as the turbidity T increases. The intensity level S_{SC} flattens out with increasing turbidity so that

$${lim}_{T \to ∞} {dS}_{SC} / dT = 0 and {lim}_{T \to ∞} {S}_{SC} ( T ) = {S}_{SCmax} .$$

In comparison, as shown by the continuous line S_{VR} in FIG. 5, the intensity level S_{VR} of detected inelastic reflections at a wavelength characteristic of the liquid medium, i.e. water Raman as for embodiments of the invention, decreases as the turbidity decreases. Here, also the intensity level S_{VR} flattens out for high turbidity values, but S_{VR} approaches 0 for T=∞. Firstly, S_{SC} flattens out at a lower NTU value (approximately 1000) than S_{VR} (approximately 10000 or even above 10000), which makes detecting S_{VR} a better measure for determining high turbidity values than S_{SC}. Secondly, it is easier to detect small differences from zero than to detect small differences from a constant value as S_{SCmax}, which also makes S_{VR} a better measure for determining high turbidity values than S_{SC}. Another difference is that dS_{VR}/dT is proportional to −1/T² for high values of NTU, whereas dS_{SC}/dT is proportional to e^{-T}. For an absorbance coefficient of 10*sample length, dS_{SC}/dT is approximately 0.00005, whereas dS_{VR}/dT is approximately 0.01, Similarly, for 20, dS_{SC}/dT is approximately 0.000000002, whereas dS_{VR}/dT is approximately 0.025. As shown, the resolution is much better for the intensity level SVR of detected inelastic reflections at a wavelength characteristic of the liquid medium than for the intensity level SSC based on detected reflected incoming light. This means that the turbidity determined from S_{VR} is more precise than the turbidity determined from S_{SC}, also for turbidities below the transition region of FIG. 3a. Observe that the scales on the y-axis may be different for S_{SC} and for S_{VR}.

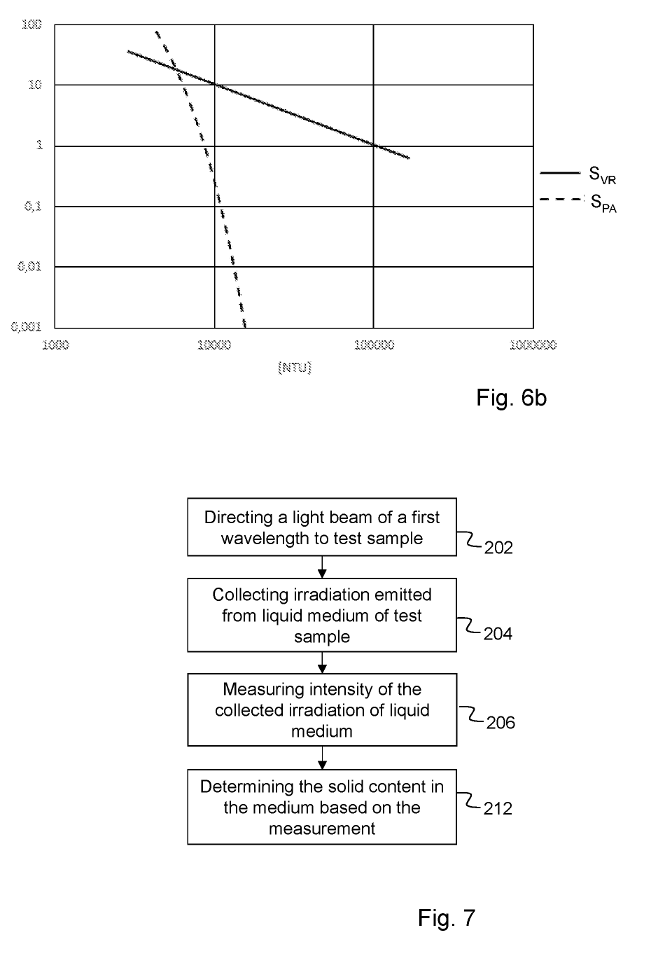

Prior art apparatuses, both probes and external apparatuses measuring solids content/turbidity are normally based on measurement of elastically scattered light at different angles. Even if dimensions are small for these apparatuses, high contents of solids will make the emitting light scatter multiple times on the way through the test sample before the light is registered by the apparatus detector. Effectively during such circumstances, such prior art apparatus acquires properties that can be described by Lambert Beer's law with a given path length. For very high turbidities the light in the sensor of the prior art apparatus will be dispersed to such an extent that a bent sample path will have the same properties as a straight one of the same length. Light in a Lambert Beer's law test sample path decreases proportionally to e^{-T} when Turbidity, T, goes to infinity. The corresponding characteristics for S_{VR} signal of the apparatus according to the invention is T⁻¹, FIGS. 6a and 6b show measured signal strength for the inventive S_{VR} (continuous line) and the prior art SPA (dotted line) as a function of NTU for small signal strength levels. Here it can be observed that there is a detectable signal strength for S_{VR} for high turbidities, as long as the detector has a high sensitivity, whereas SPA is not detectable even with a high precision detector for the same high turbidities. Observe that FIG. 6b has a logarithmic scale on the y-axis, Here, the prior art sensor discussed in FIGS. 6a and 6b has a geometrical path length of approx. 5 mm. To be able to improve the prior art apparatus, the path length needs to be shortened. However, it is difficult to lower the path length considerably in such apparatuses. Observe that the scales on the y-axis may be different for S_{VR} than for S_{PA}.

FIG. 2 shows a possible penetration depth in a sample having high absorption coefficient, i.e. high turbidity when using an apparatus as in FIG. 1. As can be seen, the incident light only reaches a short distance below the surface. As a consequence, the intensity of inelastic scattering from a test sample is lower in FIG. 2 compared to in FIG. 1, which shows possible penetration depth in a test sample having low absorption coefficient, i.e. low turbidity. When seeing the short penetration depth of the sample of high turbidity of FIG. 2, it is clear that there would be very little irradiation, if any, that would have gone through the sample, if an angle between incoming light and detected scattered irradiation of e.g. 90 degrees would have been used in an apparatus of FIG. 1. However, by arranging the apparatus of FIG. 1 such that the irradiation collected by the detector is emitted from a first area of the test sample and which first area is at least partly illuminated by the light beam of the light source, the detected scattered irradiation would become high enough also for turbid samples.

If the concentration of other substances in the test sample than the liquid medium, e.g. oil, becomes high, also the intensity level S_{VR} of detected inelastic reflections at a wavelength characteristic of the liquid medium will become lower, as the proportion of medium to other substances will be lowered. In other words, the reflected irradiation I_{H20} falling onto the detector 24 will decrease as the concentration of other substance increases. In the same way, the incident light falling onto the other substance increases with increased substance concentration, however, as the substance concentration increases, the penetration depth decreases and a possible irradiation from the other substances will flatten out at a maximum limit for further increased formula concentration of other substances.

The diagrams of FIGS. 3a-b show experimental calculations for measured intensity of water Raman irradiation S_{VR} from a test sample when used in an apparatus such as the apparatus of FIG. 1. FIG. 3a shows measured intensity S on the y-axis in relation to a certain measured turbidity in NTU on the x-axis. In FIG. 3a there is an insignificant volume share of other substances in the test sample, in relation to the amount of water. The dotted line shows measured turbidity in NTU in relation to measured Raman intensity. The marked transition region is where the test sample path length will no longer conform the limitation of scattered light on detector, when the turbidity is further increased. The dashed line S_{VR[1/T]} describes a straight line estimation of the relationship between measured turbidity and measured intensity in the transition region. The dashed-dotted line S_{VR[Error]} describes an estimation of the measurement errors. The errors increase for very high turbidities as the intensity signal becomes very weak. Similarly, for very low turbidities the intensity signal becomes strong but the measurement precision is bad as small calibration variations results in large measurement errors. There in between, the measurement of water Raman can be used with a high precision for determining turbidity of the test sample. FIG. 3a also shows the calibration value S_{VR[CALIB]} according to the first calibration step, from a reference sample that is clean, i.e. having no turbidity, and the calibration value S_{S[CALIB]} according to the second calibration step, from a reference sample that has a turbidity at the transition region.

FIG. 3b shows measured water Raman intensity, S_{VR} on the y-axis and volume share of substance in the test sample in relation to whole volume for a significant volume share of other substances in the test sample. In the figure, 0.2 means 20% substance, 1 means 100% substance in the test sample. The different lines show Water Raman signal strength S_{VR} for substances having different absorption index at 100% concentration. The dotted line shows a substance having absorption index 0.01 at 100% concentration, the continuous line a substance having absorption index 10 at 100% concentration, and so on. As shown, the water Raman signal dips quicker for higher absorption index. For insignificant volume shares of other substances as well as for significant volume shares of other substances, calibration of the apparatus can be made in approximately the same region, i.e. in the transition region, i.e. at S_{VR}=100-150.

FIG. 4 shows another embodiment of an apparatus for determining solids content of a liquid medium of a test sample. This apparatus has a zero degree angle between incident light Iₒ and reflected light Iᵣ, as could be seen from the figure. This is achieved by the use of dichroic mirrors, Dichroic mirrors are arranged to reflect certain wavelengths while other wavelengths passes through the mirror. However, other technologies similar to dichroic mirrors for achieving incident and reflected light having a mutual angle of zero degrees may also be used. The apparatus of FIG. 4 comprises a light source 102 in the shape of e.g. a solid state laser that emits light of a certain wavelength or wavelength range. The apparatus further comprises a first mirror 106 that may be adjustable and that is arranged to reflect light originating from the light source 102 towards a first dichroic mirror 108, The first dichroic mirror 108 is arranged to reflect light reflected from the first mirror 106 that is of a first wavelength range so that the light of the first wavelength range falls as incident light I₀ onto a test sample 140 comprising a liquid medium in which there is a substance. Light of wavelengths outside the first wavelength range passes through the first dichroic mirror 108, The apparatus may further have an optional neutral density filter 104 and/or a line filter arranged between the first mirror 106 and the first dichroic mirror 108. The neutral density filter and the line filter are arranged to filter out undesired wavelengths before the desired first wavelength range is reflected in the first dichroic mirror.

The incident light is then inelastically and possibly also elastically reflected by the liquid medium and a possible substance in the test sample 140. The inelastic reflections are characteristic for the materials in the sample, i.e. for the liquid medium and the possible substance, which means that the inelastic reflections have a different wavelength than the first wavelength range of the incident light, if the first wavelength range is selected to be outside the sample characteristic wavelengths. Elastic reflections are mainly the reflections of the laser beam, i.e. having the first wavelength range of the incident light I₀. The first dichroic mirror 108 receives reflected irradiation from the sample and since it is arranged to let wavelengths different than the first wavelength range through, it will let the reflected irradiation due to inelastic reflection through while any possible elastic reflection having the first wavelength range is reflected by the mirror 108. The apparatus then further comprises a blocking filter 110 that is arranged to block wavelengths that are not to be analyzed by the apparatus but let wavelengths characteristic for the inelastic reflection of the liquid medium through. The wavelengths let through the blocking filter are reflected by a second mirror 112 towards an irradiation detector 125 of the apparatus. The irradiation detector 125 comprises a blocking filter 119 to filter out any wavelengths of the light falling into the irradiation detector 125 outside the second wavelength. The filtered light I_{H20r} of the second wavelength then ends up in a photomultiplier tube, PMT, 20 that detects the incoming irradiation intensity, or level. A PMT is adapted to detect low irradiation levels, such as the levels from Raman reflection and fluorescence. The detector 125 may also be a spectrophotometer of some type detecting energies at the required wavelength.

The test sample of FIG. 4 is shown as being in a bowl or similar that is filled with the test sample. However, the test sample may be brought into the apparatus in the same way as was performed in the apparatus of FIG. 1, i.e. as a free falling jet falling from a pipe 10 into a funnel 12. In a similar way, the detecting part 20 of the apparatus of FIG. 1 may be used together with a bowl or similar that is filled with the test sample, as in the FIG. 4 apparatus.

Crosstalk may occur in the apparatus of some of the embodiments described. Crosstalk signifies that some signals that are outside the one or more second wavelengths reaches the detector anyhow and are therefore wrongly detected by the detector. A compensation of such crosstalk can be achieved by inserting a second detector into the apparatus of the invention, which second detector would detect the signals at these crosstalk wavelengths. Then the measurements of the detector may be compensation for by the measurements of the second detector.

FIG. 7 is a flow chart describing a method for determining solids content of a liquid medium of a test sample. The method comprises directing 202 a light beam of a first wavelength range towards the test sample. The method further comprises collecting 204 irradiation emitted from the liquid medium of the test sample as a result of the light beam directed towards the test sample, the irradiation being collected at one or more second wavelengths that are characteristic for the liquid medium, and measuring 206 intensity of the irradiation collected at the one or more second wavelengths, Thereafter, the method determines 212 the solids content in the liquid medium of the test sample based on the measured intensity of the irradiation collected at the one or more second wavelengths. Further, the irradiation collected at the one or more second wavelengths may be emitted from a first area of the test sample, which first area is at least partly illuminated by the light beam of the first wavelength range.

Although the description above contains a plurality of specificities, these should not be construed as limiting the scope of the concept described herein but as merely providing illustrations of some exemplifying embodiments of the described concept. It will be appreciated that the scope of the presently described concept fully encompasses other embodiments which may become obvious to those skilled in the art, and that the scope of the presently described concept is accordingly not to be limited. Reference to an element in the singular is not intended to mean “one and only one” unless explicitly so stated, but rather “one or more.” All structural and functional equivalents to the elements of the above-described embodiments that are known to those of ordinary skill in the art are expressly incorporated herein and are intended to be encompassed hereby. Moreover, it is not necessary for an apparatus or method to address each and every problem sought to be solved by the presently described concept, for it to be encompassed hereby. In the exemplary figures, a broken line generally signifies that the feature within the broken line is optional.

1. An apparatus for determining solids content of a liquid medium of a test sample, the apparatus comprising:
one or more light source for directing a light beam of a first wavelength range towards the test sample;
one or more detector for collecting irradiation emitted from the liquid medium of the test sample as a result of the light beam directed towards the test sample, the irradiation being collected at one or more second wavelengths that are characteristic for the liquid medium, and for measuring an intensity of the irradiation collected at the one or more second wavelengths, and
a determining unit for determining the solids content of the liquid medium based on the measured intensity of the irradiation collected at the one or more second wavelengths.

2. The apparatus according to claim 1, wherein the apparatus is arranged such that the irradiation collected by the one or more detector is emitted from a first area of the test sample and which first area is at least partly illuminated by the light beam of the one or more light source.

3. The apparatus according to claim 2, wherein the one or more light source is arranged to illuminate an illumination area of the test sample, and the one or more detector is arranged such that the first area is substantially covered by the illumination area.

4. The apparatus according to claim 1, wherein the apparatus is arranged so that there is an angle between the light beam directed towards the test sample and the irradiation emitted from the test sample that is received by the one or more detector that is lower than 45 degrees, preferably lower than 10 degrees, most preferably approximately zero degrees.

5. The apparatus according to claim 1, wherein the one or more detector is further adapted to collect irradiation and measure intensity of the irradiation at the second wavelength, the irradiation being emitted from a first reference sample comprising a known solids content of the liquid medium, and the determining unit is adapted to determine the solids content of the liquid medium of the test sample based on the measured intensity of the irradiation collected at the second wavelength from the first reference sample as well as from the test sample.

6. The apparatus according to claim 5, wherein the one or more detector is further adapted to collect irradiation and measure intensity of the irradiation at the second wavelength, the irradiation being emitted from a second reference sample comprising a known solids content of the liquid medium different from the known solids content of the first reference sample, and the determining unit is adapted to determine the solids content of the liquid medium of the test sample based on the measured intensity of the irradiation collected at the second wavelength from the first reference sample, from the second reference sample as well as from the test sample.

7. The apparatus according to claim 1, further comprising a temperature sensor for detecting the temperature of the test sample, and wherein the determining unit is arranged for determining the solids content of the liquid medium of the test sample further based on the detected temperature of the test sample.

8. The apparatus according to claim 1, wherein the liquid medium is water and the second wavelength characteristic for the liquid medium may be a wavelength characteristic for Raman reflection of water.

9. A method for determining solids content of a liquid medium of a test sample, the method comprising:
directing a light beam of a first wavelength range towards the test sample;
collecting irradiation emitted from the liquid medium of the test sample as a result of the light beam directed towards the test sample, the irradiation being collected at one or more second wavelengths that are characteristic for the liquid medium;
measuring an intensity of the irradiation collected at the one or more second wavelengths;
determining the solids content of the liquid medium of the test sample based on the measured intensity of the irradiation collected at the one or more second wavelengths.

10. The method according to claim 9, wherein the irradiation collected at the one or more second wavelengths is emitted from a first area of the test sample and which first area is at least partly illuminated by the light beam of the first wavelength range.
